(19) United States
(12) Patent Application Publication
(10) Pub. No.: US 2024/0150696 A1
(43) Pub. Date: May 9, 2024

(54) Tapping Device for Microfluidic System

(71) Applicant: Javelin Biotech, Inc., Woburn, MA (US)

(72) Inventors: Shiny Amala Priya Rajan, Woburn, MA (US); Jacob Dylan Freake, Somerville, MA (US); Corbin Munn, New York, NY (US); Murat Cirit, Cambridge, MA (US)

(21) Appl. No.: 18/503,912

(22) Filed: Nov. 7, 2023

Related U.S. Application Data
(60) Provisional application No. 63/423,192, filed on Nov. 7, 2022.

(51) Int. Cl. C12M 3/06; C12M 1/00; C12M 1/26; C12M 1/36

(52) U.S. Cl. CPC C12M 23/16; C12M 29/14; C12M 29/24; C12M 33/00; C12M 41/48

(57) ABSTRACT

This disclosure describes hardware for microfluidic chips and an associated platform for facilitating operation of one or more microfluidic chips. The microfluidic chips described herein are designed for supporting multiple different tissue types, including kidney tissue, liver tissue, adipose cells, and so forth. Chip geometry facilities fluid flow through one or more channels of the chip with a particular flow rate. For example, shear forces are reduced where needed to ensure proper flow rate of fluid in the channels. The chamber geometry and the geometry of the channels ensures that a desired amount of oxygen is delivered to sample cells or tissues in a controlled manner.

### CLAIM OF PRIORITY

This application claims priority under 35 U.S.C. § 119(e) to U.S. Patent Application Ser. No. 63/423,192, filed on Nov. 7, 2022, the entire contents of which are hereby incorporated by reference.

### TECHNICAL FIELD

This disclosure generally relates to microphysiological systems (MPSs). More specifically, this disclosure relates to hardware systems to support and control operations of one or more MPSs either individually or in different combinations with one another. This disclosure relates to hardware designs of a plurality of MPSs for operation either individually or in combination with one another and the hardware support system.

### BACKGROUND

A microphysiological system (MPS) (also called a microfluidic chip) includes an interconnected set of two- or three-dimensional cellular constructs that are frequently referred to as organs-on-chips, tissue chips, or in vitro organ constructs. The constructs are typically made with immortalized cell lines, primary cells from animals or humans, or organ-specific cells derived from naïve cells, human embryonic stem cells, and induced pluripotent stem cells (iPSCs). Individually, each construct can be designed to recapitulate the structure and function of a human organ or organ region, paying particular attention to the cellular microenvironment and cellular heterogeneity. When coupled together to create an MPS, these constructs offer the possibility of providing, in vitro, an unprecedented physiological accuracy for the study of cell-cell, drug-cell, drug-drug, and organ-drug interactions, if drug delivery can be properly modeled.

### SUMMARY

This disclosure describes hardware for microfluidic systems (also called microfluidic chips or simply chips in this disclosure). The microfluidic chips include hardware that houses tissue constructs to provide biomimetic cues for long-term culture and physiologically-relevant tissue functions. The microfluidic chips described herein are designed for supporting multiple different tissue types, including lung tissue, kidney tissue, liver tissue, and so forth. Chip geometry facilities fluid flow through one or more channels of the chip with a particular flow rate.

A fixture to provide a mechanical impulse to a chip to dislodge dead cells attached to the bottom surface of the cell area. The fixture is designed to allow a user to provide a repeatable impulse to the chip by actuating one or both spring arms. The spring arms are actuated by pulling back on a tab and then letting go causing them to quickly move and collide with the side of the chip. The fixture keeps the chip in a consistent position during this actuation with a locating bracket.

The fixture may hold any well plate format with SBS size from 6 to 1536 well plates. The bottom of the fixture can be hollow or made of a transparent material to visualize cells with a microscope.

The microfluidic chips and hardware support module enable one or more of the following technical advantages.

The tapping device(s) of the fixture enable a user to apply a specified amount of force to the MPS to loosen and/or dislodge cells (e.g., dead cells) from a surface of a cell chamber of the microfluidic chip. The fixture keeps the microfluidic chip fixed in place so that functioning of the microfluidic loop of the chip is not disrupted. The fixture(s) can be positioned at different positions around or on the microfluidic chip to apply force to precise location(s) on the microfluidic chip. The tapping is configured to avoid disturbing healthy cells in the cell chamber.

The one or more advantages are enabled by one or more of the following implementations.

In an aspect, a microfluidic chip system includes a microfluidic chip. The microfluidic chip includes a pump configured to pump a fluid media through a fluid circuit. The microfluidic chip includes a cell chamber in the fluid circuit, the cell chamber supporting a plurality of cells, the cell chamber configured to receive the fluid media including oxygen for exposing the plurality of cells to the fluid media including the oxygen. The microfluidic chip includes a re-oxygenation chamber configured to add oxygen to the fluid media circulating in the fluid circuit. The system includes a platform that includes a bracket for receiving the microfluidic chip. The platform includes at least one tapping device, the tapping device configured for actuation to mechanically contact the microfluidic chip any apply a force to the microfluidic chip, the force being sufficient to loosen cells from an inner surface of the fluid circuit.

In some implementations, the at least one tapping device includes an arm that extends from a hinge, the hinge being coupled to the platform. In some implementations, the at least one tapping device includes a tapping head at an end of the arm, the tapping head configured to contact the microfluidic chip when the microfluidic chip is seated in the platform. In some implementations, the arm pivots around the hinge to move the tapping head to contact the microfluidic chip.

In some implementations, there are two tapping devices including the at least one tapping device. In some implementations, a first tapping device, of the two tapping devices, is configured to contact a first side of the microfluidic chip. A second tapping device of the two tapping devices is configured to contact a second side of the microfluidic chip. In some implementations, the first side is orthogonal to the second side.

In some implementations, the platform further includes markings that specific a distance for moving the at least one tapping device, the distance corresponding to an amount of force for contacting the microfluidic chip with the at least one tapping device.

In some implementations, the platform comprises a stopper that prevents the at least one tapping device from applying a force to the microfluidic chip that is over a threshold amount of force.

In some implementations, the at least one tapping device comprises a plunger. In some implementations, the plunger is in communication with a linear actuator configured to cause the plunger to move and tap the microfluidic chip. In some implementations, the microfluidic chip comprises a switch configured to enable sampling or disable sampling of the fluid media of the fluid circuit. In some implementations, the system includes a controller configured to automatically actuate the at least one tapping device.

In an aspect, a microfluidic chip platform includes a bracket for receiving a microfluidic chip. The microfluidic chip platform includes at least one tapping device, the tapping device configured for actuation to mechanically contact the microfluidic chip any apply a force to the microfluidic chip, the force being sufficient to loosen cells from an inner surface of a fluid circuit of the microfluidic chip when the microfluidic chip is received within the bracket.

In some implementations, the at least one tapping device includes an arm that extends from a hinge, the hinge being coupled to the platform. In some implementations, the at least one tapping device includes a tapping head at an end of the arm, the tapping head configured to contact the microfluidic chip when the microfluidic chip is seated in the platform. In some implementations, the arm pivots around the hinge to move the tapping head to contact the microfluidic chip. In some implementations, there are two tapping devices including the at least one tapping device. In some implementations, a first tapping device, of the two tapping devices, is configured to contact a first side of the microfluidic chip, and wherein a second tapping device, of the two tapping devices, is configured to contact a second side of the microfluidic chip.

In some implementations, the platform includes markings that specify a distance for moving the at least one tapping device, the distance corresponding to an amount of force for contacting the microfluidic chip with the at least one tapping device.

In some implementations, the platform includes a stopper that prevents the at least one tapping device from applying a force to the microfluidic chip that is over a threshold amount of force.

In some implementations, the at least one tapping device comprises a plunger. In some implementations, the plunger is in communication with a linear actuator configured to cause the plunger to move and tap the microfluidic chip. In some implementations, the platform includes a controller configured to automatically actuate the at least one tapping device.

The details of one or more embodiments of these systems and methods are set forth in the accompanying drawings and the description to be presented. Other features, objects, and advantages of these systems and methods are apparent from the description and drawings, and from the claims.

### BRIEF DESCRIPTION OF THE DRAWINGS

FIG. 1 illustrates a top view of an example microfluidic chip that is positioned on a support device including one or more tapping devices.

FIG. 2 illustrates a perspective view of an example of a platform for a microfluidic chip.

FIG. 3 illustrates a top view of an example of a platform for a microfluidic chip.

FIGS. 4A-4B each illustrate a side view a perspective view of an example of a platform for a microfluidic chip.

FIG. 5 illustrates a bottom view of an example of a platform for a microfluidic chip.

FIG. 6 shows a perspective view of a microfluidic system including the platform of FIG. 2.

FIG. 7 shows a top view of the microfluidic system of FIG. 6.

FIG. 8 shows a top view of a microfluidic system.

### DETAILED DESCRIPTION

This disclosure describes microfluidic chips configured for recirculation of a fluid medium and re-oxygenation of cell cultures within the microfluidic chips. This disclosure describes hardware support systems for controlling recirculation of the fluid medium and re-oxygenation of the cell cultures within the microfluidic chips. A MPS can be referred to as organ construct house in a microfluidic chip and organ on chips (OOC). In some implementations, the cell chambers of the microfluidic chip are called compartments. In the microfluidic chips described herein, the microfluidic chip generally includes a fluid circulation loop for recirculating fluid through a cell chamber including cell tissue of a given cell type or types. Generally, the fluid is continuously recirculated through the cell chamber in a closed loop. The fluid loop generally includes a re-oxygenation chamber to re-oxygenate oxygen-depleted fluid medium recirculating through the fluid loop, as the oxygen is absorbed by the cells of cell chamber. The microfluidic chip design can be based on the oxygen requirements of the cells in the cell chamber and the maximum shear stress tolerances and oxygen absorption rates of those cells, which can limit flow rates of the fluid medium.

The microfluidic chip is seated in a hardware platform that connects to the microfluidic chip and controls functionality of the microfluidic chip, such as recirculation of fluid media in a fluid loop. Examples of the microfluidic chips are described in International Pat. Ser. No. PCT/US2022/075223, filed on Aug. 19, 2022, titled MICROFLUIDIC SYSTEMS AND SUPPORT MODULE, the entire contents of which are incorporated by reference herein.

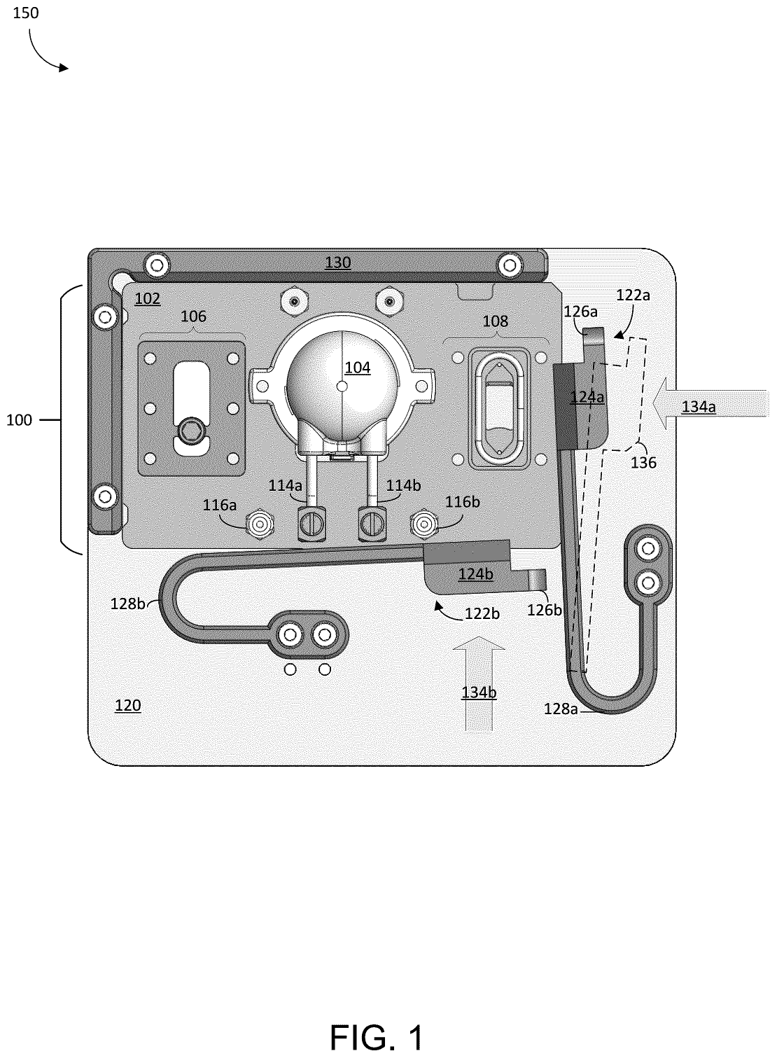

FIG. 1 illustrates a top view of an example microfluidic system 150 including a microfluidic chip 100 in a hardware platform 120. The microfluidic chip 100 is configured to emulate organ functionality. The tissue that can be hosted may include liver tissue, adipose tissue, kidney tissue, muscle tissue, or most any tissue type. Generally, depending on the tissue type, a geometry of a cell chamber hosting the tissue is adjusted as described herein. The microfluidic chip 100 is configured to enable preclinical drug discovery and assist in developing treatments for various diseases. The microfluidic chip 100 enables long-term tissue mono- or co-culture (e.g., longer than 4 weeks). The microfluidic chip 100 includes a substrate 102 in which various components, such as a pump 104 (e.g., a peristaltic pump) is mounted for circulating fluid through inserts such as a re-oxygenation chamber 106 and a cell chamber 108.

The microfluidic chip 100 is configured for being seated in a hardware platform 120. The hardware platform includes a controller and a user interface for controlling operation of the pump 104 and recirculation of the fluid media in the microfluidic chip 100. The hardware platform is configured to connect to the on-chip controller of the microfluidic chip 100.

The hardware platform includes tapping device(s) 120a-b. The tapping devices 120a-b are configured to physically tap the microfluidic chip 100. The tapping devices 120a-b tap the microfluidic chip 100 to remove dead cell(s) that are stuck to surfaces in the fluid loop of the microfluidic chip 100. For example, dead cells can be stuck in a channel or in the cell chamber 108 of the fluid loop. To loosen these cells, the tapping device(s) 120a-b can be actuated.

The tapping devices 120a-b each include a head 124a-b on the end of an arm 128a-b. The arms 128a-b are semi-flexible and each form a respective spring. For each tapping device 122a-b, a tab 126a-b on the head 124a-b can be pulled back by a user to tension the spring formed by the arm 128a-b and released. The head 124a-b snaps back to the resting position and taps on the side of the microfluidic chip 100. Dotted line 136 shows an example of a tensioned tapping device 122a prior to release. Arrows 134a-b show direction to tapping by the devices 122a-b.

The physical tapping knocks cells (e.g., dead cells) from inner surfaces of the fluid loop. As shown in FIG. 1, there are two tapping device 122a-b, a device 122a on the end of the microfluidic chip 100 and a device 122b on the side of the microfluidic chip 100. However, any configuration of one or more tapping devices 122 can be possible. While U-shaped springs are shown (e.g., made of a semi-rigid plastic or metal), other actuation device are possible, such as plungers, leaf springs, cantilevers, and so forth, as subsequently described.

The platform 120 for supporting the microfluidic chip 100 includes a bracket 130 that is sized to the shape of the microfluidic chip 100. The bracket 130 holds the microfluidic chip 100 in place when the tapping devices 120a-b are actuated. The tapping devices 120a-b therefore contact the microfluidic chip 100 to loosen the dead cells.

The microfluidic chip 100 includes microfluidic passages forming a fluid loop. The pump 104 is included to circulate the fluid media through the fluid loop. The pump 104 has ports 112a-b for fluid intake and output. Access ports 116-b enable access to the fluid loop (e.g., for adding/removing fluid media). In some implementations, a channel feature (not shown) is added to smooth flow from the peristaltic pump 104.

The microfluidic chip 100 includes a fluid circulation loop configured to re-circulate fluid in the microfluidic chip through the cell chamber 108 including tissue that is the subject of organ emulation or other experimentation. Generally, the fluid medium is continuously recirculated through the cell chamber 108 in a closed loop. The fluid loop generally includes a re-oxygenation chamber 106 to re-oxygenate oxygen-depleted fluid medium recirculating through the fluid loop, as the oxygen is absorbed by the cells of cell chamber 116. The geometry of the cell chamber 116, loop, re-oxygenation chamber 106, etc. is generally based on the oxygen level requirements of the liver or adipose cells in the cell chamber 116. The fluid loop includes the pump 104, the re-oxygenation chamber 106, the cell culture chamber 108, and input and output ports.

The re-oxygenation chamber 106 is configured to enable oxygen to mix with the fluid to reintroduce oxygen at specified concentrations to the fluid medium of the fluid loop. A permeable membrane of the chamber 106 enables air to enter the chamber and mix with the fluid medium, dissolving in the fluid medium and re-oxygenating the fluid medium. A sampling port (not shown) enables samples of the fluid medium to be removed from the fluid loop for analysis. For example, the oxygen concentration, drug concentration, etc. of the fluid medium can be measured by extracted fluid through the sampling port. This can enable analysis of how much oxygen or how much of a drug has been absorbed by the cells of the cell chamber 108.

The pump 104 is configured to pump the fluid medium from the re-oxygenation chamber 106 to the valve 114. The valve 114 is configured to change positions (as subsequently described) to enable fluid flow through the valve 114 from the channel 112b to the input port 144 of the cell chamber 116. The valve enables fluid to flow from the exit port 150 of the cell chamber 116 to the channel 112a or to the channel 152 that leads to the flow sensor 102. The flow sensor 102 is configured to sense a flow rate of fluid in the fluid loop. The flow sensor data is accessible by port 132. Fluid flowing through the flow sensor 102 flows through channel 154 back to the re-oxygenation chamber 106 to complete the fluid loop. The flow sensor 102 is coupled to the substrate 118 by a flow sensor housing 130a.

The cell chamber 108 includes tissue that can be subjected to drugs or other materials in the fluid for emulation of tissue functionality. For example, the cell chamber 108 can include most any tissue type. The cell source can be human primary cells, stem cells or cell lines. Alternatively, primary cells and/or immortalized cell line be used as sources for tissue in the microfluidic chip 100. Generally, primary cells are cells that have been isolated and then used relatively quickly (e.g., immediately) or after cryopreservation. Diseased cells representing one or more stages of a disease can be introduced into the cell chambers of the microfluidic chip 100.

The microfluidic chip 100 is configured for disease modeling by enabling emulation of tissues in controlled environments. For performing a test there can be an induction of disease in the cells of the microfluidic chip 100 in the prepared liver and/or adipose tissues. The disease progression can be monitored over long periods of time. In some implementations, the microfluidic chip 100 can include cells from diseased patients. Many different mechanisms can be used for on-chip disease induction, as subsequently described.

The microfluidic chip 100 is configured for disease characterization. For example, multi-scale assays can be performed using the microfluidic chip 100. The microfluidic chip 100 enables evaluation of cell construct and tissue construct functions. The microfluidic chip 100 enables a comparison of healthy phenotypes and disease phenotypes. The microfluidic chip 100 enables acquisition of data from pre-determined phenotypic metrics and -omics analysis.

A computing system (not shown) uses data developed from the MPS 100 to combine MPS models with model-informed drug discovery (MIDD) methodologies in a processing workflow. The processing workflow, described in relation to U.S. patent application Ser. No. 17/104,708, filed on May 27, 2021, incorporated in entirety by reference herein, can be used to develop an understanding of disease diagnostic and response biomarkers. The computational modeling is performed for target (e.g., drugs or drug combinations) discovery using the MPS data of the MPS 100 and systems biology (SB) and quantitative systems pharmacology (QSP) based models. These models are configured to identify molecular abnormalities for diseased cells or tissues. The models link the molecular data to the phenotypic data. Here, phenotypic data can include clinical information regarding disease symptoms, as well as relevant demographic data (if applicable), such as age, ethnicity, and sex.

The microfluidic chip 100 is configured for drug target discovery and drug development. Each platform 120 combines a tissue-engineered MPS “on-chip” model. Data generated by analyzing the behavior of the tissue of the microfluidic chip 100 can be used to establish metabolically dysfunctional (MetS) MPSs and spheroid models for the liver and adipose tissue of a human. In an example, the primary human parenchymal and non-parenchymal liver cells can be sourced from a single donor for all aims and experiments to minimize the risk of adverse allograft interactions (e.g. Kupffer cell allo-antibodies). Metabolic dysfunction can be induced by culturing the microfluidic chip 100 in a defined, serum-free medium containing disease-relevant concentrations of glucose, fructose, insulin, FFAs, and TNF-α (multi-hit medium (MHM)) and then compared to microfluidic chips cultured in a physiologically healthy medium (PHM). Details of this analysis are described in U.S. patent application Ser. No. 17/104,708, filed on May 27, 2021, incorporated in entirety by reference herein.

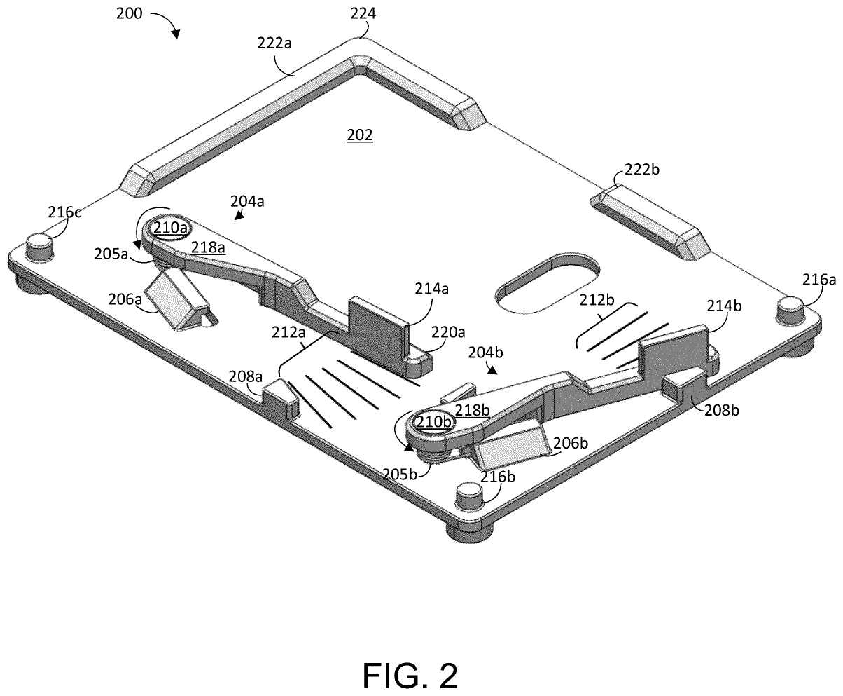

FIG. 2 shows an example of a platform 200 for a microfluidic chip, such as the platform 120 for microfluidic chip 100 of system 150 of FIG. 1. The platform 200 includes tapping devices 204a-b that are mounted on a substrate 202. The platform 200 is compatible with any type of microfluidic chip that is sized to fit on the substrate 202. Specifically, microfluidic chips hosting different tissue types or cell types can be used with the platform 200. For example, a microfluidic chip with one fluid loop is compatible with the platform 200. A microfluidic chip with two or more fluid loops is compatible with the platform 200. The hardware platform 200 includes standoffs, such as standoff 216, for resting on other surfaces.

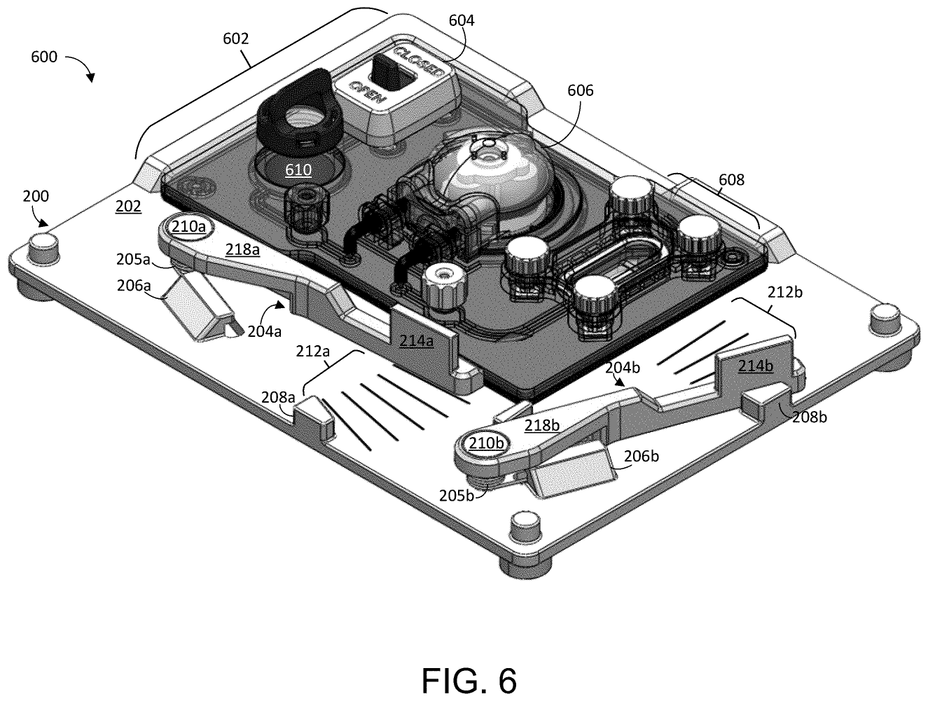

As previously described, the tapping devices 204a-204b are configured to impact the microfluidic chip (e.g., chip or chip 602 shown in FIG. 6) with a particular amount of force. The force imparted by the tapping devices 204a-b on the chip 100 is configured to release cells or tissue that is attached to walls of microfluidic channels in a fluid loop of the microfluidic device. Generally, the cells or tissue are dead and may clog the microfluidic channels or impede fluid flow within the channels. The microfluidic hip 100 is configured to cause recirculation of fluid media within the fluid loop at a finely tuned flow rate because the oxygen levels within the cell chamber of the microfluidic chip are maintained at precise levels. The exact rate of fluid flow depends on a type of the microfluidic chip, and thus a type of cells or tissue hosted in the microfluidic device. The tapping devices 204a-b help a user to remove dead tissue from the flow path in the fluid loop by knocking the cells or tissue free from walls of one or more portions of the fluid loop.

The tapping devices 204a-b are each coupled to the platform substrate 202. Tapping devices 204a and 204b can operate in a similar manner. Focusing first on tapping device 204a, the tapping device 204a includes an arm 218a that extends along the substrate 202. The tapping device can be coupled to the hardware platform substrate 202 using a hinge 210a. The hinge 210a can be spring loaded using a mechanical spring 205a. The spring 205a is actuated by pulling back on a tab 214a that is at the end of the arm 218a. Pulling back on the tab 214a, stores potential energy in the spring 205a, which then is released when the tab 214a is related. A tapping head 220a is configured to contact the microfluidic chip and impart a force on the chip, releasing kinetic energy into the chip. The force is configured to cause cells or tissue that is stuck to walls or the chambers or channels of the chip to release from the walls of the chambers or channels. In some implementations, the hinge 210a (or another coupling mechanism) is actuated using a motor (such as a servo motor or other similar motor). The motor can cause the arm 218a to swing and contact the chip using the tapping head 220a.

The tapping device 204a is configured to contract the chip with a controlled amount of force. The amount of force can be configured based on the geometry of the tapping device 204a. For example, the arm 218a can be longer to enable a greater amount of force to be imparted. A mass of the tapping head 220a can be increased to increase the momentum of the tapping device 204a and increase a tapping force of the tapping device. In this example, the arm is at least two centimeters long up to 15 centimeters long. Depending on a size of the microfluidic chip, the arm 218a can be longer or shorter than 2-15 centimeters.

The spring 205a of the tapping device 204a can be actuated by pulling the arm 218a backward towards the stopper 208a. The spring is coupled to the surface 202 inside of the spring housing 206a. The amount of force of the tapping head 220a on the microfluidic chip is tunable based on how far back the arm 218a is pulled toward the stopper 208a. For a maximum force, the arm 218a is pulled all the way back to the stopper 208a. An area on the substrate 202 includes ruled markings 212a (e.g., a ruler or graduated area). The markings 212a represent tuned values for application of different forces on the microfluidic chip. Each of the markings 212a can represent a particular amount of force that is applied to the microfluidic chip when the arm 218a is pulled back to that point. For example, the first marking can represent a first force, the second marking represents a second, slightly larger force, and so forth. In some implementations, each of the markings 212a represents a preferred force for a respective chip. A user can pull tab 214a back to a marking of the markers 212a a release the tab. The arm 218a snaps back to tap the microfluidic chip using the tapping head 220a with a particular force. This ensures that the cells or tissue are not damage by applying too much force to the microfluidic chip, but that enough force is applied to free cells or tissue from walls of chambers or channels of the chip. The markings 212a ensure that a consistent amount of force is applied to the chip with different applications of the tapping force.

The tapping device 204a is configured to tap a first side of the microfluidic chip. The first side can be a longer side of the chip. The platform includes a second tapping device 204b that is configured to tap a second side of the microfluidic chip. The second side can be a shorter side orthogonal to the first side.

The second tapping device 204b is configured to operate in a manner similar to the first tapping device 204a. The tapping device 204b includes an arm 218b that is similar to arm 218a and coupled to the substrate 202 at a pivot point 210b. The tapping device 204b includes a spring 205b that is similar to spring 205a. The tapping device 204b includes a spring housing 206b similar to spring housing 205a. The tapping device 204b includes a tab 214b that is similar to tab 214a. A stopper 208b is configured to allow the arm 218b to be pulled back to a specific maximum distance, similar to stopper 208b. Ruled markings 212b enable a measurement of the amount of force being applied by the tapping device 204b, similar to markings 212a for tapping device 204a as previously described.

While two tapping devices 204a, 204b are shown, the platform 200 can include additional tapping devices for tapping other side of the microfluidic chip. In some implementations, the platform omits either tapping device 204a or tapping device 204b and only one tapping device is used.

The tapping devices 204a-b can be manual devices that are actuated by a user or automatic devices that are configured for communication with a controller. For example, as shown, the tapping devices 204a-b can be pulled back and released by a user for application of the tapping force on the microfluidic device. In some implementations, one or more of the tapping devices 204a-b is in communication with a controller (not shown) configured to actuate the tapping device 204a-b with a control signal. For example, the controller can generate an electrical signal to cause a motor (or another electronic device) to actuate and/or move the tapping device. In some implementations, the controller can be in communication with one or more sensors configured to measure a presence or number of cells or amount of tissue that is stuck to walls of channels or chambers of the microfluidic chip. When the cells or tissue presence exceeds a threshold amount as sensed by the one or more sensors, the controller can cause the activation of the tapping devices. For example, the sensor can include a beam sensor (e.g., an infrared sensor) that measures an opacity of a chamber wall or channel by emitting a beam through the wall. When cells build up on the wall, the radiation is blocked, and the blockage is detected by the controller. Other such sensors can be used with the microfluidic chip.

The tapping devices 204a, 204b are shown with similar configurations in FIG. 2. However, the tapping devices 204a-b can have different configurations if needed. For example, one of the tapping devices 204a, 204b can be larger or smaller than the other, use a different actuation mechanism (e.g., either manual or automated), apply different forces, or have an entirely different configuration.

One or both of the tapping devices 204a-b can be configured with one or more other manual or automatic devices for tapping the microfluidic chip. For example, the tapping devices 204a-b can include hammers, gears, levers, cantilevers, or other mechanisms for tapping the microfluidic chip with at a controlled force. The actuation devices can include electromagnets, leaf springs, linear springs, or other such devices configured to cause the mechanical device to move with a controlled force.

A bracket includes walls 222a and 222b that are configured to contact the microfluidic chip 602 and hold the microfluidic chip in place when the microfluidic chip is struck by the tapping device 204a and/or device 204b. Wall 222a has a corner 224 to support the chip in two directions on the surface of the platform 200. The wall 222b supports the chip when struck by the tapping device 204a.

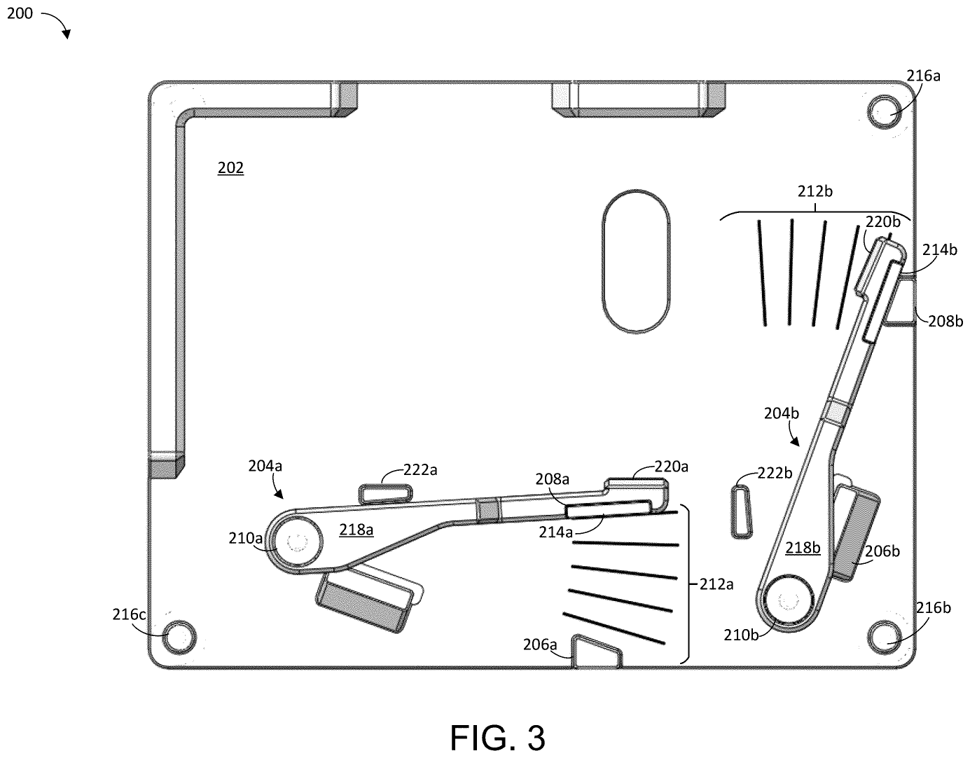

FIG. 3 shows a top view of the platform 200 of FIG. 2. In FIG. 3, the tapping head 220b of the second tapping device 204b is shown. The tapping device 204a is configured to pivot in a vertical direction around hinge 210a and across markings 212a. The tapping device 204b is configured to pivot around hinge 210b and move in a horizontal direction across markings 212b. Tapping device 204a is shown in a resting state and tapping device 204b is shown in an activated state, pulled back to maximum range at stopper 208b.

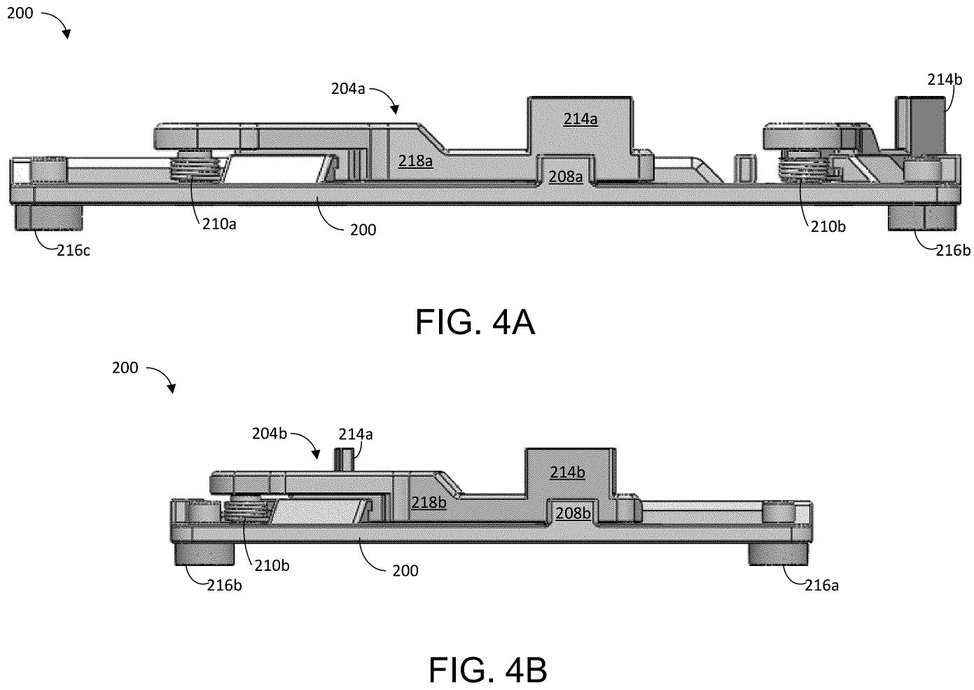

FIGS. 4A-4B show side views of the platform 200 of FIG. 2. FIG. 4A shows a view along a side edge of the platform. FIG. 4B shows a view along a front edge of the platform. In FIG. 4A, the tab 214a of the tapping device 204a is shown behind the stopper 208a. The arm 218a extends from the spring 210a to the tab 214a. In FIG. 4B, the tab 214b of the tapping device 204a is shown behind the stopper 208b. The arm 218b extends from the spring 210b to the tab 214b. Standoffs 216a-c are visible.

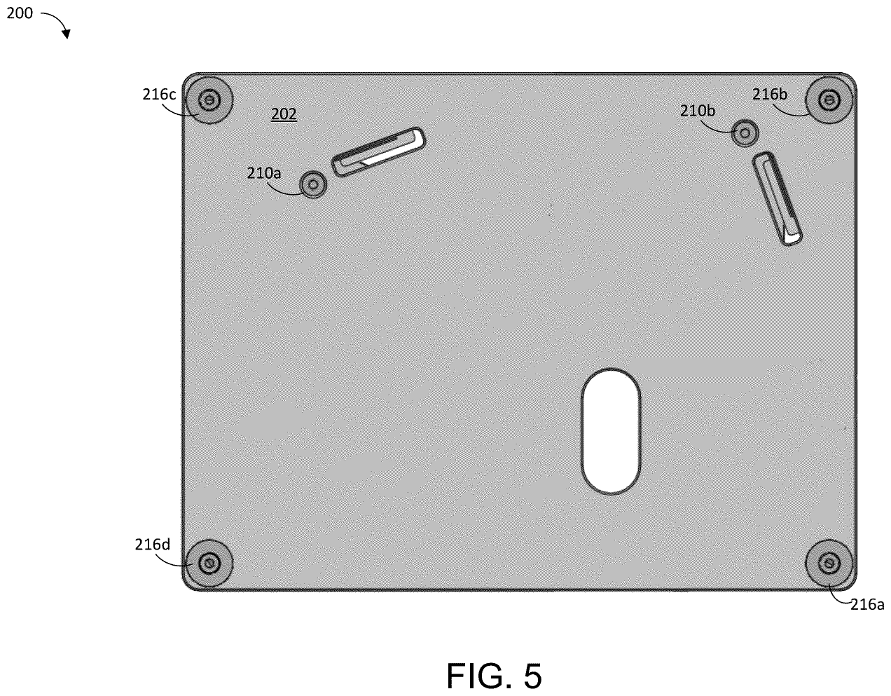

FIG. 5 shows a bottom view of the platform 200 of FIG. 2. Standoffs 216a-d are visible attached to substrate 202. Hinges 210a-210b are visible through the substrate 202.

FIG. 6 shows a perspective view of a microfluidic system 600 including the platform 200 of FIG. 2. A microfluidic chip 602 is shown as seated on the platform 200. The microfluidic chip 602 can be similar to the microfluidic chip 100 described in relation to FIG. 1. The microfluidic chip 602 includes a pump 606, a cell chamber 608, a sampling chamber 610, and a sampling switch 604. The microfluidic chip 602 can be removed from the platform 200 and replaced with another instance of the microfluidic chip.

The switch 604 of the microfluidic chip 602 is configured to open or close a path in the fluid loop through the sampling chamber 610 of the microfluidic chip. The switch can include a sliding switch that can be toggled to an open position or a closed position. When the switch 604 is in the open position, the fluid media is diverted through the sampling chamber 610. When the switch 604 is in the closed position, the fluid media bypasses the sampling chamber 610 in the fluid loop. In some implementations, the switch includes a valve. In some implementations, the switch includes a shutter system that blocks the channel to the sampling chamber 610.

The tapping device 204a is configured to tap a first side of the microfluidic chip 602. The tapping device 204b is configured to tap a second side of the microfluidic chip 602. The tapping devices 204a-b are configured to loosen cells or tissue that are stuck to the walls of the cell chamber 608 or channels of the microfluidic chip 602.

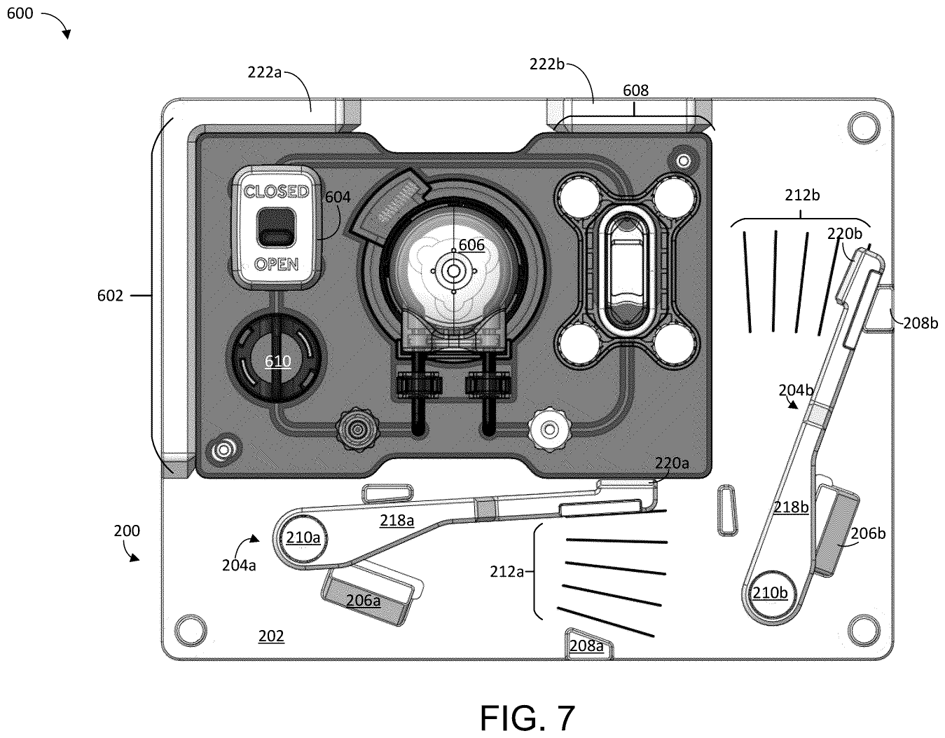

FIG. 7 shows a top view of the microfluidic system 600 of FIG. 6. Tapping head 220a of tapping device 204a is shown as contacting the microfluidic chip 602. The tapping device 204b is retracted and ready to be released to contact the microfluidic device 602 with tapping head 220b. Walls 222a and 222b are configured to contact the microfluidic chip 602 and hold the microfluidic chip in place when the microfluidic chip is struck by the tapping device 204a and/or device 204b.

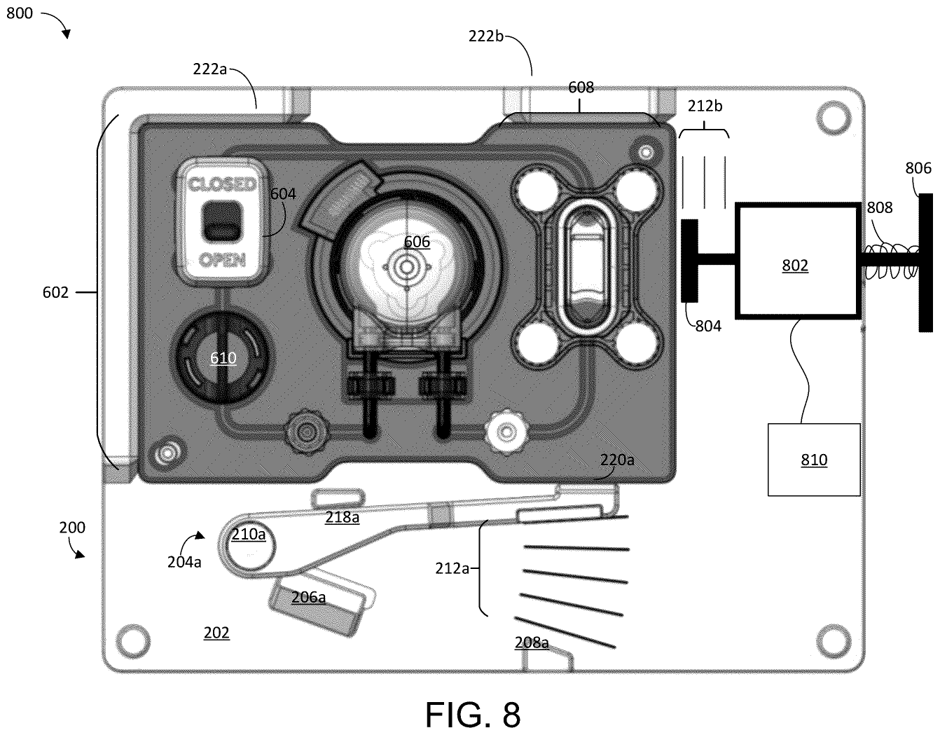

One or both of the tapping devices 204a-b can be changed from a pivoting arm to a plunger, leaf spring, or other configuration. In an example, one or both of the tapping devices 204a-b can include a linear plunger that is actuated with an electrical signal or manually by a user, as shown in FIG. 8. In system 800, the user can pull back a plunger 806 to compress a spring 808 and release the plunger to enable a plunger head 804 to contact the microfluidic chip 602. The plunger 806 can have markings 212b to enable measurement of the amount of force to be applied to the chip. A body 802 of the plunger device can fasten the plunger to the platform 200. In another example, the plunger 806 can be actuated using an electrical signal from a controller 810 that controls operation of the microfluidic chip 602. A capacitor can be used to store an amount of charge. When a force is to be applied, the capacitor is discharged, the current can be used to actuate the device (such as a plunger 806) to impart the force using the tapping head 804. In some implementations, the controller 810 can similarly be configured to control the tapping devices 204a-b. In some implementations, the controller 810 is not on the platform 200 but is in a support module that is remote from the platform.

Reference is made in detail to implementations, examples of which are illustrated in the accompanying drawings. In the previous description, numerous specific details are set forth in order to provide a thorough understanding of the various described implementations. However, it are apparent to one of ordinary skill in the art that the various described implementations may be practiced without these specific details. In other instances, well-known methods, procedures, components, circuits, and networks have not been described in detail so as not to unnecessarily obscure aspects of the implementations.

Several features are described that can each be used independently of one another or with any combination of other features. However, any individual feature may not address any of the problems discussed above or might only address one of the problems discussed above. Some of the problems discussed above might not be fully addressed by any of the features described in this specification. Although headings are provided, data related to a particular heading, but not found in the section having that heading, may also be found elsewhere in this description.

Accordingly, the previously described example implementations do not define or constrain the present disclosure. Other changes, substitutions, and alterations are also possible without departing from the scope of the present disclosure. Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

What is claimed is:

1. A microfluidic chip system, comprising:
a microfluidic chip comprising:
a pump configured to pump a fluid media through a fluid circuit;
a cell chamber in the fluid circuit, the cell chamber supporting a plurality of cells, the cell chamber configured to receive the fluid media including oxygen for exposing the plurality of cells to the fluid media including the oxygen; and
a re-oxygenation chamber configured to add oxygen to the fluid media circulating in the fluid circuit; and
a platform comprising:
a bracket for receiving the microfluidic chip; and
at least one tapping device, the tapping device configured for actuation to mechanically contact the microfluidic chip any apply a force to the microfluidic chip, the force being sufficient to loosen cells from an inner surface of the fluid circuit.

2. The system of claim 1, the at least one tapping device comprising:
an arm that extends from a hinge, the hinge being coupled to the platform;
a tapping head at an end of the arm, the tapping head configured to contact the microfluidic chip when the microfluidic chip is seated in the platform;
wherein the arm pivots around the hinge to move the tapping head to contact the microfluidic chip.

3. The system of claim 1, further comprising two tapping devices including the at least one tapping device.

4. The system of claim 3, wherein a first tapping device, of the two tapping devices, is configured to contact a first side of the microfluidic chip, and wherein a second tapping device, of the two tapping devices, is configured to contact a second side of the microfluidic chip.

5. The system of claim 4, wherein the first side is orthogonal to the second side.

6. The system of claim 1, the platform further comprising markings that specifies a distance for moving the at least one tapping device, the distance corresponding to an amount of force for contacting the microfluidic chip with the at least one tapping device.

7. The system of claim 1, wherein the platform comprises a stopper that prevents the at least one tapping device from applying a force to the microfluidic chip that is over a threshold amount of force.

8. The system of claim 1, wherein the at least one tapping device comprises a plunger.

9. The system of claim 8, wherein the plunger is in communication with a linear actuator configured to cause the plunger to move and tap the microfluidic chip.

10. The system of claim 1, wherein the microfluidic chip comprises a switch configured to enable sampling or disable sampling of the fluid media of the fluid circuit.

11. The system of claim 1, further comprising a controller configured to automatically actuate the at least one tapping device.

12. A microfluidic chip platform, comprising:
a bracket for receiving a microfluidic chip; and
at least one tapping device, the tapping device configured for actuation to mechanically contact the microfluidic chip any apply a force to the microfluidic chip, the force being sufficient to loosen cells from an inner surface of a fluid circuit of the microfluidic chip when the microfluidic chip is received within the bracket.

13. The platform of claim 12, the at least one tapping device comprising:
an arm that extends from a hinge, the hinge being coupled to the platform;
a tapping head at an end of the arm, the tapping head configured to contact the microfluidic chip when the microfluidic chip is seated in the platform;
wherein the arm pivots around the hinge to move the tapping head to contact the microfluidic chip.

14. The platform of claim 12, further comprising two tapping devices including the at least one tapping device.

15. The platform of claim 14, wherein a first tapping device, of the two tapping devices, is configured to contact a first side of the microfluidic chip, and wherein a second tapping device, of the two tapping devices, is configured to contact a second side of the microfluidic chip.

16. The platform of claim 12, further comprising markings that specifies a distance for moving the at least one tapping device, the distance corresponding to an amount of force for contacting the microfluidic chip with the at least one tapping device.

17. The platform of claim 12, further comprising a stopper that prevents the at least one tapping device from applying a force to the microfluidic chip that is over a threshold amount of force.

18. The platform of claim 12, wherein the at least one tapping device comprises a plunger.

19. The platform of claim 18, wherein the plunger is in communication with a linear actuator configured to cause the plunger to move and tap the microfluidic chip.

20. The platform of claim 12, further comprising a controller configured to automatically actuate the at least one tapping device.
